(19) United States
(12) Patent Application Publication
(10) Pub. No.: US 2023/0293068 A1
(43) Pub. Date: Sep. 21, 2023

(54) EXTRACTION CONTAINER

(71) Applicant: LumiraDx UK Ltd., London (GB)

(72) Inventors: Remus Haupt, San Diego, CA (US); Keerthi Boddupally, San Diego, CA (US)

(21) Appl. No.: 17/992,774

(22) Filed: Nov. 22, 2022

Related U.S. Application Data
(63) Continuation-in-part of application No. PCT/US2021/034073, filed on May 25, 2021.
(60) Provisional application No. 63/082,246, filed on Sep. 23, 2020.
(60) Provisional application No. 63/069,549, filed on Aug. 24, 2020.
(60) Provisional application No. 63/029,579, filed on May 25, 2020.

(51) Int. Cl. A61B 5/15; A61B 10/00; G01N 33/569; G01N 1/44

(52) U.S. Cl. CPC A61B 5/150351; A61B 10/0051; G01N 1/44; G01N 33/56911

(57) ABSTRACT

An extraction container includes a molded tubular body having openings at opposing first and second end thereof. A base cap is configured to obstruct a base opening disposed at the base of the tubular body. The base cap is integrally molded with the tubular body and connected thereto by a frangible connection. The base cap can be removed by breaking the frangible connection to de-obstruct the second opening. The first base cap and tubular body include integrally molded engagement features to permit the base cap to be reattached to the tubular body to re-obstruct the base opening. A removable foil or polymer seal obstructs a top opening disposed at the top end of the tubular body. A top cap and hinge are integrally molded with the tubular body and configured to re-obstruct the top opening following removal of the seal.

### CROSS REFERENCE TO RELATED APPLICATIONS

This application claims the benefit of and priority to U.S. Provisional Application No. 63/082,246 filed Sep. 23, 2020; U.S. Provisional Application No. 63/069,549 filed Aug. 24, 2020; U.S. Provisional Application No. 63/029,579 filed May 25, 2020, and International Application No. PCT /US2021/034073 filed May 25, 2021 titled “Extraction Container”, each of which is incorporated by reference herein in its entirety.

### FIELD OF THE INVENTION

The present invention relates to extraction containers useful for extracting sample material from a sample collection device such as a swab.

### BACKGROUND OF THE INVENTION

Determining the presence of a target in a biological sample may include the following steps. A sample collector, e.g., a swab, is used to collect a sample, such as a sample of saliva, mucous, sputum, and/or blood. The sample collector is then introduced into a vial containing an extraction liquid such as a buffer or lysing medium to remove some of the collected sample from the collection device and form a mixture with the extraction liquid. The resulting mixture is subjected to a diagnostic determination, e.g., an immunoassay, to determine the presence of the target in the biological sample.

In many cases, the target is a pathogen such that the biological sample constitutes a biohazard. It is important that a user be able to manipulate the extraction container containing the biological sample without contaminating the user or their surroundings.

It is also important that diagnostic kits used in determining targets be manufacturable rapidly and at low cost to permit widespread accessibility to such kits in times of need such as during an outbreak of disease caused by a particular pathogen.

### SUMMARY OF THE INVENTION

Disclosed herein, in embodiments, is a method comprising: removing a removable seal sealing an access opening of a generally tubular body thereby exposing a liquid disposed within the generally tubular container; combining a sample comprising a target with the exposed liquid thereby forming a liquid mixture of the sample and the liquid within the generally tubular body; resealing the access opening with a lid comprising (i) a dispensing opening and (ii) a securing component that obstructs the dispensing opening and is secured to the lid via a frangible connection; with the access opening resealed with the lid, heating the liquid mixture within the generally tubular container to a temperature of at least about 1001Zl C, wherein the lid and the securing component essentially prevent liquid of the liquid mixture or gas within the generally tubular body from leaking or escaping therefrom during the heating; and after heating the liquid mixture, opening the dispensing opening by breaking the frangible connection between the securing component and the lid and then dispensing at least some of the liquid mixture from the generally tubular body through the dispensing opening.

In embodiments, the generally tubular container and the lid including the securing component are made of a polymer. In embodiments, the securing component is a cap and the method further includes resecuring the cap to the lid to reseal the dispensing opening after dispensing the at least some liquid mixture. In embodiments, the method further includes subjecting the at least some dispensed liquid mixture to an assay to determine the presence and/or amount of the target. In embodiments, the target is indicative of a pathogen.

In embodiments, the step of heating comprises subjecting the liquid mixture within the generally tubular body to microwave radiation. In embodiments, the step of heating comprises heating the liquid mixture to a temperature and for a duration sufficient to lyse bacterial cells within the liquid mixture. In embodiments, the step of heating comprises heating the liquid mixture to a temperature and for a duration sufficient to lyse bacterial cells of Mycobacterium tuberculosis and/or a bacterium of the genus Streptococcus. In embodiments, the target is indicative of at least one of Mycobacterium tuberculosis and/or a bacterium of the genus Streptococcus and the method further includes subjecting the at least some dispensed liquid mixture to an assay to determine the presence and/or amount of the target. In embodiments, the heating step comprises heating the liquid mixture for a period of less than about 200 seconds and more than about 60 seconds. In embodiments, the heating step comprises heating the liquid mixture to a temperature that is at least about 115° C. and less than about 150° C.

In embodiments, the total volume of the liquid mixture within the tubular body during the heating step is less than about 50 µL. In embodiments, during the heating step, no more than about 0.5% of the total mass of the liquid mixture present at the start of the heating step escapes or leaks from the tubular body. In embodiments, during the heating step, less than about 0.25% of the total mass of the liquid mixture present at the start of the heating step escapes or leaks from the tubular body.

In embodiments, the step of dispensing comprises squeezing an exterior surface of the tubular body between fingers of a user to increase a gas pressure therein and dispense the at least some liquid mixture through the dispensing opening. In embodiments, the lid comprises a filter and the step of dispensing comprises passing the at least some liquid mixture through the filter. In embodiments, the liquid within the tubular body comprises at least one of buffer, universal transport media (UTM), Viral Transport Media (VTM) or lysis medium. In embodiments, the sample comprises at least one of saliva, mucous, sputum, or blood. In embodiments, the removable seal comprises one or more layers comprising a liquid-impermeable metal foil and/or a liquid-impermeable polymer film. In embodiments, an interior surface of the tubular body defines a multifoil.

In embodiments, an extraction container includes a generally tubular body including first and second opposed ends, the first end defining a first opening having a first maximum internal diameter and the second end defining a second opening having second maximum internal diameter smaller than the first maximum diameter; a filter disposed within the tubular body and disposed such that a liquid disposed within the tubular body must pass through the filter before passing through the second opening and exiting the tubular body; a cap obstructing the passage of liquid through the second opening, integrally molded with the tubular body, and secured to the tubular body via a frangible connection; and wherein an exterior surface of the tubular body includes at least one cap engagement feature and an interior surface of the cap comprises at least one container engagement feature and further wherein the frangible junction securing the cap and tubular body can be broken to permit liquid within the tubular body to pass through the filter and second opening to exit the tubular body and the at least one cap engagement feature and at least one container engagement feature can then be engaged such that the cap again obstructs the passage of liquid through the second opening thereby preventing liquid from exiting the tubular body therethrough.

In embodiments, the extraction container further includes liquid disposed within the tubular body and a removable seal disposed and configured to prevent the passage of liquid or gas through the first opening. The seal may include or be formed of a layer of metal foil and/or a layer of polymer. The extraction container may consist essentially of the tubular body, first and second caps, seal and the liquid disposed within the tubular body.

In embodiments, the cap of the foregoing extraction containers may be a first cap and the tubular body may include a second cap connected to the tubular body by a hinge and configured to reseal the first opening following the removal of the seal. The first and second caps and hinge may include, e.g., may be formed as, a single integrally molded component.

The liquid of the foregoing extraction containers may be an extraction buffer configured to facilitate the extraction of a target present in a biological sample, e.g., a sample including at least one of saliva, mucous, sputum, and blood introduced therein. For example, the extraction buffer may include a lysis reagent configured to lyse one or more cells present in the biological sample.

In embodiments, a method includes inserting a swab carrying a biological sample through a first opening of a generally tubular body of an extraction container into a liquid disposed within the tubular body of the extraction container and manipulating the swab to release at least some of the sample into the liquid, the first opening being disposed at a first end of the tubular body; after the manipulating step, removing the swab from the extraction container and then sealing the first opening; after sealing the first opening of the extraction container, breaking a frangible connection securing a cap to a second end of the tubular body of the extraction container, the cap being integrally molded with the extraction container and configured, when secured to the extraction container via the frangible connection, to prevent the passage of liquid through the second opening thereby preventing liquid from exiting the tubular body therethrough; with the frangible connection broken, increasing a gas pressure within the tubular body of the extraction container to cause at least some of the liquid disposed therein to pass through a filter disposed within the extraction container and then exit the tubular body of the extraction container through the second opening; and after causing at least some of the liquid to exit the tubular body of the extraction container, engaging at least one cap engagement feature disposed on an exterior surface of the extraction container adjacent the second opening with at least one container engagement feature disposed on an interior surface of the cap such that the cap again obstructs the passage of liquid through the second opening thereby preventing liquid from exiting the tubular body therethrough.

In embodiments of the foregoing method, the step of causing at least some of the liquid to exit the tubular body of the extraction container through the second opening further includes applying at least some of the exiting liquid to a sample application port of a diagnostic device configured to determine the presence of a target in the biological sample. The target may be a pathogen or an antibody or antigen indicative of the presence of a pathogen in the biological sample. The pathogen may be a respiratory pathogen such as an influenza virus, a respiratory syncytial virus, or a coronavirus, e.g., Covid-19. The pathogen may be a bacterium such as Mycobacterium tuberculosis or a bacterium of the genus Streptococcus.

In embodiments of the foregoing methods, the method further includes, prior to the step of inserting the swab, removing a removable seal from the extraction container, the removable seal disposed and configured to prevent the passage of liquid or gas through the first opening. The seal may include, e.g., be formed of, a layer of metal foil and or a layer of polymer and removing a removable seal from the extraction container may include peeling the seal from the extraction container.

The cap of the extraction container of the foregoing methods may be a first cap and the tubular body may include a second cap connected to the tubular body by a hinge and the method further comprises resealing the first opening with the second cap after removing the swab from the tubular body. The tubular body, the first and second caps and hinge may include, e.g., may be formed as, a single integrally molded component. The liquid may be disposed in the tubular container prior to the step of removing the seal from the top opening thereof.

A method of manufacturing any of the extraction container may include integrally molding the tubular body, the first and second caps, and hinge; disposing the filter into the tubular body such that a liquid disposed within the tubular body must pass through the filter before passing through the second opening and exiting the tubular body; introducing the liquid into the tubular body; and sealing the first opening with the seal.

In any of the foregoing methods or extraction containers, the first and/or second engagement features may include one or more threads, one or more notches and projections, or a combination(s) thereof.

In embodiments, the method of using the extraction container may include subjecting the liquid within the extraction container to a heating step, e.g., to a temperature and for a duration as disclosed herein. For example, the heating may be performed by subjecting the liquid within the tubular body to microwave radiation. In some cases, the heating step is performed prior to the step of breaking the frangible cap connection to open the second opening to permit liquid to be dispensed from the extraction container through the second opening, e.g., to perform an analysis of one or more targets therein. Accordingly, in some cases, after the heating step, the frangible connection is broken to permit liquid to be dispensed from the extraction container.

In embodiments, a method includes forming a mixture including at least a portion of each of one or more samples (e.g., specimens) obtained from each of a plurality of mammals; and subjecting the mixture to an immunological assay to determine the presence or absence of at least one target therein. The presence of the target in at least one of the at least one or more samples obtained from a mammal is indicative of the presence of a medical condition and/or status of the mammal. The method may include detecting the presence of the target in the mixture and determining, based on such presence, that the medical condition and/or status is present in at least one of the plurality of mammals. The absence of the target in all of the at least one or more samples obtained from a mammal, e.g., the presence of the target in none of the at least one or more samples obtained from a mammal, may be indicative of the absence of the medical condition and/or status of the mammal. The forgoing methods may include detecting the absence of the target in the mixture and determining, based on such absence, that the medical condition and/or status is absent in all of the plurality of mammals, e.g., that the medical condition or status is not present in any of the plurality of mammals. The target may be associated with a pathogen, e.g., the target may be an antigen such as a spike protein, a nucleocapsid protein, an envelope protein, a membrane protein, or a hemagglutinin-esterase dimer protein of a coronavirus. The antigen may be a nucleocapsid protein, such as of a coronavirus such as SARS-CoV-2. The target may be indicative of a bacterium such as Mycobacterium tuberculosis or a bacterium of the genus Streptococcus. The medical condition may be an infectious disease and/or the status may be having an infectious disease such as COVID-19, tuberculosis, or strep throat.

In the foregoing methods, each of the at least one samples from each of the plurality of mammals may include blood, serum, plasma, saliva, mucus, and/or a specimen collected from a throat, nasopharyngeal or nasal swab. The target may a coronavirus, e.g., SARS-CoV-2, and (i) the step of subjecting the mixture to an immunological assay, comprises binding an antibody, or fragment thereof, to a receptor binding domain (RBD) of a coronavirus spike protein, or a fragment thereof, of the coronavirus or (ii) wherein the target is an antigen, e.g., a spike protein, a nucleocapsid protein, an envelope protein, a membrane protein, or a hemagglutinin-esterase dimer protein of the coronavirus. The step of subjecting the at least a portion of a mixture to an immunological assay may include applying the at least a portion of the mixture a microfluidic device disclosed in the ‘782 application including as claimed in any of claims 72- 89, 107-123, or 140-156 thereof and using such microfluidic device to subject the at least a portion of the mixture to the immunological assay. The step of subjecting may be performed using an instrument to operate the microfluidic device as disclosed or claimed in the ‘782 application. Each of the at least one samples from each of the mammals may include a specimen collected from a throat, nasopharyngeal or nasal swab. The method may further include, for each of the at least one samples from each of the mammals, introducing a head of each swab containing the specimen into a volume of buffer, universal transport media (UTM), Viral Transport Media (VTM) or lysis medium. The method may include, for each of the at least one samples from each of the mammals, maintaining at least the head of each swab in the volume of buffer, UTM, or VTM when introducing the head of each subsequent swab into the volume of buffer, UTM, VTM or lysis medium. The method may include, for each of the at least one samples from each of the mammals, after the step of introducing: separating at least a portion of the swab including the head of the swab from the remainder of the swab. The method may include forming the mixture in any of the extraction containers as disclosed or claimed herein.

The step of forming the mixture may include forming the mixture including at least the portion of each of one or more samples obtained from each of a number N mammals, wherein the number N is at least about 2, at least about 3, at least about 4, at least about 5, at least about 6, at least about 7, at least about 8, at least about 9, or at least about 10. The step of forming the mixture, may include forming the mixture including at least the portion of each of one or more samples obtained from each of a number N mammals, wherein the number N is about 25 or less, about 20 or less, about 15 or less, about 10 or less, about 9, or less, about 8, or less, about 7 or less, about 6 or less, or about 5 or less. Each mammal of the plurality of mammals may be a human being.

In embodiments, a method for detecting a coronavirus antigen in a mixture including a sample from each of a plurality of subjects includes subjecting the sample to a binding assay including a first and a second reagent, wherein the first reagent comprises an antibody to a coronavirus antigen, wherein the first reagent is labeled with a detectable label or a capture agent, and wherein the second reagent is attached to a detectable label or a capture agent, and wherein the first reagent and the second reagent can bind the coronavirus antigen to form a complex including the first reagent, the coronavirus, and the second reagent. Performing the step of subjecting the sample to a binding assay may include performing the binding assay according to any of claims 125-139 of the ‘782 application.

The mixture may include at least the portion of each of one or more samples obtained from each of a number N subjects, wherein the number N is at least about 2, at least about 3, at least about 4, at least about 5, at least about 6, at least about 7, at least about 8, at least about 9, or at least about 10. The mixture may include at least the portion of each of one or more samples obtained from each of a number N subjects, wherein the number N is about 25 or less, about 20 or less, about 15 or less, about 10 or less, about 9, or less, about 8, or less, about 7 or less, about 6 or less, or about 5 or less.

In embodiments, an extraction container includes a generally tubular body defining a longitudinal axis and including (i) a closed end and an open end spaced apart from one another along the longitudinal axis and (ii) an interior surface, wherein at least a portion of the interior surface is spaced-apart radially from the longitudinal axis and defines a multifoil. The multifoil may be a trefoil, quatrefoil, cinquefoil, sexfoil, septfoil, or octofoil.

The generally tubular body of the extraction container may have a total length along the longitudinal axis between a location on the interior surface of the extraction container at the closed end and the opening of the extraction container, and wherein a ratio of the total length along the longitudinal axis of the portion of the interior surface that defines a multifoil to the total length of the extraction container is at least about 0.15, 0.175, 0.2, 0.225, or 0.25. At least a portion of the interior surface of the extraction container that defines a multifoil may define a plurality of radii about the longitudinal axis and wherein a ratio of a minimum radius of the radii to a maximum radius of the radii is about 0.95 or less, about 0.9 or less, about 0.85 or less, or about 0.8 or less. The interior surface that defines the radii may define a number Nmin minimum radii wherein Nmin > 2. In embodiments, the number Nmin is the number of the multifoil. The interior surface that defines the radii may define a number Nmax minimum radii wherein Nmax > 2. In embodiments, Nmax is the number of the multifoil. In embodiments, Nmin = Nmax. Each of the Nmin may be at least substantially the same as or essentially the same as the others of the Nmin. Each of the Nmax is at least substantially the same as or essentially the same as the others of the Nmax. In embodiments, the interior surface that defines a multifoil defines a number NL lobes, NL is the number of the multifoil, and the interior surface defining each lobe is arcuate. The interior surface defining each lobe may be circular or elliptical.

The extraction container may further include a liquid and a plurality of collection swab tips disposed therein. The number of collection swab tips disposed in the extraction container may be equal to the number of the multifoil.

The total length along the longitudinal axis of the portion of the interior surface of the extraction container that defines a multifoil may be at least about as long as, e.g., about the same length as, a length of the collection swab tip, e.g., the collection swab tip of a swab, or plurality of swabs, included in a kit with the extraction container. Each collection swab tip may include at least a portion of a biological sample collected from a different respective mammalian subject. Each biological sample may include a nasal or a nasopharyngeal sample. Each mammalian subject may a human. At least a portion of the interior surface that defines a multifoil may be disposed adjacent the closed end of the generally tubular body.

The extraction container may further include a liquid disposed within the generally tubular member and a removable liquid impermeable seal sealing the open end thereof. The liquid may include a buffer, e.g., a viral transport medium, or lysis buffer. The seal may include or be formed as a metallic foil, polymer film, or combination thereof. The liquid may occupy a depth of at least about 5 mm, at least about 7.5 mm, at least about 10 mm, or at least about 12.5 mm within the generally tubular member when the tubular member is positioned with the longitudinal axis generally vertical and the open end oriented about the closed end. Substantially all of the inner surface of the generally tubular member that is contacted by the liquid may include or consist of the portion of the interior surface that defines a multifoil. In embodiments, at least some, substantially all, or essentially all of the interior surface that defines a multifoil is in contact with the liquid.

The extraction container may further include a lid configured to close the open end. The lid may include a passage configured, when in an open state, to permit liquid within the generally tubular body to pass through the passage and exit the tubular body and a cap secured to the lid via a frangible connection and disposed to obstruct the passage, wherein, when the cap is removed by breaking the frangible connection, the passage is opened. The cap and lid may each include at least one respective engagement feature that can be engaged such that the cap again obstructs the passage.

In embodiments, a kit includes any of the foregoing extraction containers and a plurality of sample collection swabs, with each sample collection swab optionally having a sample collection swab tip. The extraction container of the kit may include a liquid, e.g., a buffer, VTM, and/or lysis medium, and a seal closing the opening of the extraction container.

In embodiments, an extraction container includes a generally tubular body including a lower end and an upper end; a liquid disposed within the lower end of the tubular body; a passage extending from within the lower end of the tubular body to the exterior of the lower end of the tubular body; a cap secured to the tubular body and configured in a secured state to prevent the liquid from exiting the tubular body through the passage, wherein the cap may be released to open the passage and permit the liquid to exit the tubular body through the passage; and one or more collection swab tips at least partially immersed within the liquid.

In embodiments, the number of collection swab tips at least partially immersed within the liquid is at least 2 and less than about 8. The number of collection swab tips at least partially immersed within the liquid may be, e.g., 3, 4, 5, or 6. Each collection swab tip comprises a biological sample obtained from a respective different mammal, e.g., from a respective different human being. Each collection swab tip may be respectively secured to a portion of a different collection swab shaft portion. Each collection swab shaft portion may include a remaining portion of a frangible shaft connection that was broken by a user thereby separating a portion of the swab shaft including the swab tip from a remainder of the swab shaft. The upper end of the tubular body may include an opening, and optionally further include a lid secured to and closing the opening at the upper end. The cap may be integrally molded with the tubular body and secured thereto via a frangible cap connection. In embodiments, breaking the frangible cap connection releases the cap to open the passage and permit the liquid to exit the tubular body through the passage. The cap and the exterior of the lower end of the tubular body may include at least one engagement feature that permits the cap to be resecured to the tubular body.

A method of using the extraction container may include releasing the cap to open the passage and causing at least some of the liquid to exit the tubular body through the passage. The step of causing at least some of the liquid to exit the tubular body through the passage may be performed while the collection swab tips are in the liquid within the tubular body. The step of causing at least some of the liquid to exit the tubular body through the passage may include applying at least some of the liquid that has exited the tubular body to a sample application zone of a diagnostic device. The method of using the extraction container may further include using the diagnostic device to perform a diagnostic assay for at least one target present in the liquid. The target may be indicative of the presence of a pathogen, e.g., a coronavirus. The target may be indicative of a bacterium such as Mycobacterium tuberculosis or a bacterium of the genus Streptococcus. The diagnostic device may be a microfluidic device disclosed, e.g., claimed, in the ‘782 application.

The method of using the extraction container may include subjecting the liquid within the extraction container to a heating step, e.g., to a temperature and for a duration as disclosed herein. For example, the heating may be performed by subjecting the liquid within the tubular body to microwave radiation. The heating step is performed prior to the step of breaking the frangible cap connection to open the passage to permit liquid to be dispensed from the extraction container, e.g., to perform an analysis of one or more targets therein. After the heating step, the frangible connection is broken to permit liquid to be dispensed from the extraction container.

In embodiments, a method may include providing a number N collection swabs, where N is at least 2 and each collection swab comprises a collection swab tip including a biological sample obtained from a respective different mammalian subject; for each of the N collection swabs, inserting at least the collection swab tip of the collection swab through an opening of an extraction container into a liquid disposed therein and leaving at least the collection swab tip in the extraction container; and closing the opening of the extraction container with the liquid and N collection swab tips disposed therein. The method may include combining the liquid and at least some of the biological sample from each of the N collection swab tips to form a mixture. In embodiments, the volume of the liquid, prior to the insertion of the first of the N collection swab tips therein, is about 1250, microliters or less, about 1000 microliters or less, about 900 microliters or less, about 800 microliters or less, about 750 microliters or less, about 700 microliters or less, or about 600 microliters or less. The liquid may include a buffer such as VTM, UTM, lysis medium or combination thereof.

In embodiments, the method may further include removing at least a portion of the mixture and, optionally, determining the presence of one or more targets in at least a portion of the removed portion of the mixture. The target may be indicative of the presence of a pathogen, e.g., a coronavirus. The determining the presence of the one or more targets may include subjecting the removed portion of the mixture to a binding assay disclosed in the ‘782 application. The subjecting the removed portion of the mixture to a binding assay disclosed in the ‘782 application may include subjecting the mixture to a binding assay claimed in the ‘782 application, e.g., a binding assay according to any of claims 125-139 of the ‘782 application.

In embodiments, the opening is a first opening and when the extraction container is positioned in a first orientation, the first opening is disposed above the mixture within the extraction container and the method further comprises, with the extraction container disposed in the first orientation, removing the at least the portion of the mixture through a second opening of the extraction container disposed below the mixture. The extraction container may include a cap secured to the tubular body and configured in a secured state to prevent the liquid from exiting the tubular body through the passage, wherein the cap may be released to open the passage and permit the liquid to exit the tubular body through the passage when the extraction container is in the first orientation.

In embodiments, an extraction container includes a generally tubular bodying defining a longitudinal axis and including a first opening and a second opening spaced apart from one another along the longitudinal axis; a removable seal covering the first opening; a lid configured to close (e.g., reseal) the first opening after the seal has been removed; a cap obstructing the passage of liquid through the second opening, integrally molded with the tubular body, and secured to the tubular body via a frangible connection; and a liquid disposed within the tubular body. The lid may be integrally molded with the extraction container and attached thereto. The diameter of the first opening is larger than the diameter of the second opening. For example, the first opening is larger to permit, e.g., access for swabs or other samples to be inserted into and removed from the generally tubular body. The second opening is smaller and facilitates liquid within the container to be dispensed drop-wise. The minimum diameter of the first opening may be at least about 10 mm and the minimum diameter of the second opening is about 2 mm or less, e.g., between about 0.5 mm and about 2 mm. The seal may include, e.g., be formed as, a layer of metal foil and/or a layer of polymer. The seal may be peelably removable.

In embodiments, the frangible connection securing the cap to the tubular body may be broken to permit liquid within the tubular body to pass through the second opening to exit the tubular body. An exterior surface of the tubular body may include at least one cap engagement feature and an interior surface of the cap comprises at least one container engagement feature and, after the frangible connection is broken, the at least one cap engagement feature and at least one container engagement feature can then be engaged such that the cap again obstructs the passage of liquid through the second opening thereby preventing liquid from exiting the tubular body therethrough.

A method of using the extraction container may include removing the removable seal and combining one or more biological samples with the liquid in the tubular body. Removing the removable seal exposes the liquid disposed within the tubular body so that the samples can be combined with the exposed liquid therein. The opening of the tubular body through which the sample(s) were inserted e.g., on a sample collection swab, is sealed with the lid. With the frangible connection in the unbroken state, the extraction container may be heated, e.g., to a temperature and for a duration as disclosed herein. For example, the heating may be performed by subjecting the liquid within the tubular body to microwave radiation. After the heating step, the frangible connection between the cap and lid may be broken to permit the mixture of the liquid and sample(s) to be dispensed, e.g., to perform an analysis of a target(s) therein as disclosed herein.

In embodiments, a method includes heating a liquid mixture, e.g., an aqueous mixture, contained within a sealed container. The container may include a cap secured thereto via a frangible connection. In the attached state, the cap may obstruct an opening of the container to essentially prevent, e.g., prevent, the aqueous mixture from leaking from the container through the opening and/or essentially prevent, e.g., prevent, a gas, e.g., vaporized components of the liquid mixture, from escaping the container through the opening during the heating. During the heating, in some cases, the sealed container does not have any opening or route that would permit the liquid mixture from leaking from the container and/or a gas, e.g., vaporized components of the mixture, from escaping the container during the heating.

The mixture may be a combination of a liquid, e.g., an aqueous liquid, and a sample. The liquid may be an aqueous liquid and include or consist essentially of, e.g., any of the liquids disclosed herein and suitable for the processing of biological samples, e.g., one or more of a buffer, universal transport media (UTM), Viral Transport Media (VTM) or lysis medium. The sample may be, e.g., a biological sample including or consisting essentially of any of the biological samples disclosed herein including e.g., at least one of saliva, mucous, sputum, tissue, and blood.

In embodiments of the method, the opening that is obstructed by the cap is a dispensing opening and the container includes a generally tubular body having a longitudinal axis and defining an access opening at one end thereof. The dispensing opening facilitates liquid within the container to be dispensed drop-wise whereas the access opening is larger to permit, e.g., swabs or other samples to be inserted into and removed from the generally tubular body. The generally tubular body includes the liquid disposed therein and a removable seal disposed and configured to prevent the passage of liquid or gas through the access opening, e.g., during transport and storage of the container. The removable seal may be, e.g., any of the removable seals disclosed herein. The container may include a lid configured to reseal the access opening once the removable seal is removed. The lid includes a dispensing passage, an entry opening and the dispensing opening spaced apart along a longitudinal axis for the dispensing passage. The cap is secured to the lid via the frangible connection.

The method may further include removing the removable seal, prior to the heating, to free the access opening and provide access to the liquid disposed within the generally tubular body. The method may further include combining the sample with the liquid disposed therein to form the liquid mixture. For example, a portion of a swab carrying the sample may be inserted into the liquid through the access opening. After, or during, the step of combining the sample with the liquid disposed therein to form the liquid mixture, the lid may be attached to seal the access opening and essentially prevent, e.g., prevent leakage of the aqueous mixture or escape of the gas through the access opening during heating as described above.

The method may include, after the step of heating, breaking the frangible connection between the cap and container, e.g., breaking the frangible connection between the cap and the lid, thereby freeing the access opening of the container. The method may include, after breaking the frangible connection, dispensing an amount of the liquid mixture from the container through the free dispensing opening. After the step of dispensing, the method may include reattaching the cap to the lid to seal the dispensing opening and essentially prevent, e.g., prevent, the leakage of the liquid mixture or gas from escaping the container. For example, the cap and/or lid may include an engagement feature as disclosed herein to secure the cap and lid.

An assay may be performed using the liquid mixture dispensed through the free opening. For example, the assay may be an assay for the determination of a target, e.g., a biomolecule such as an antigen, antibody, or protein, indicative of the presence and or amount of a pathogen present in the aqueous mixture. For example, the assay may be any of the assays disclosed or claimed in the ‘325 application, e.g., as in any of claims 124-139 or 176-214 thereof. The assay may be performed using any of the microfluidic devices and systems disclosed or claimed in the ‘325 application, e.g., in any of claims 140-156. The dispensing step may include dispensing the aqueous sample to a diagnostic device, e.g., to a sample application port of the diagnostic device. The diagnostic device may be, e.g., any of the aforementioned microfluidic devices.

By essentially preventing the liquid mixture from leaking or essentially preventing the gas, e.g., vaporized components of the mixture, from escaping the container during heating, the cap in the attached state permits no more than 1%, no more than 0.5%, no more than 0.25%, or essentially none of the total mass of the mixture to leak or escape the container during heating. The lid provides the same functionality with respect to preventing the liquid mixture and/or gas from leaking or escaping via the access opening.

For any embodiment described herein, the heating may be performed, e.g., by subjecting the liquid mixture within the container to electromagnetic radiation, e.g., microwave radiation and/or infrared radiation. Alternatively, or in combination with subjecting the liquid mixture to electromagnetic radiation, the heating may be performed by placing the sealed container in thermal communication with a heat source such as a heating block or other form of heat exchanger.

The heating is typically sufficient to lyse cells, e.g., cells of one or more pathogens and/or cells of the subject, e.g., a mammal such as a human, obtained with the sample. For example, the heating may be sufficient to lyse cells of bacteria such as Mycobacterium tuberculosis or a bacterium of the genus Streptococcus. The heating may include heating the aqueous mixture to a temperature of at least about 105° C., at least about 110° C., at least about 115° C., at least about 120° C., at least about 125° C., at least about 130° C., or at least about 135° C. The heating may include heating the aqueous mixture to a temperature of about 175° C. or less, about 0165 Cor less, or about 0150 Cor less. The heating, e.g., the heating to any of the aforementioned temperatures may occur for a period of at least about 60 seconds, at least about 70 seconds, at least about 80 seconds, at least about 90 seconds, at least about 100 seconds, at least about 110 seconds, or at least about 120 seconds. The heating, e.g., the heating to any of the aforementioned temperatures may occur for a period of about 250 seconds or less, about 200 seconds or less, about 180 seconds or less, about 160 seconds or less, about 150 seconds or less, about 130 seconds or less, or about 120 seconds or less.

The method may include monitoring the temperature of the aqueous mixture during heating. In some embodiments, any container described herein further comprises a temperature sensor physically and/or operatively coupled thereto. The temperature monitoring may be performed by infrared monitoring, a temperature sensor in thermal communication with an exterior of the container, and/or a temperature sensor disposed within an interior of the container. In embodiments, the interior of the container is free of any temperature sensor.

Components of the extraction containers disclosed herein, e.g., a generally tubular body, lid, and/or cap, may be formed of a polymer material such as polypropylene, polyethylene, or cycloolefin. Components of the extraction containers may be integrally formed. For example, a generally tubular body and lid with cap may be integrally formed. In some embodiments, a generally tubular body is formed of a first polymer that is sufficiently pliable to permit a user to squeeze the generally tubular body when dispensing a sample. For example, a user may squeeze an exterior surface of the tubular body between fingers of the user to increase a gas pressure therein and dispense the sample. A lid with a cap secured thereto by a frangible connection may be formed of a second, more rigid polymer to provide a more secure seal during a heating step.

In any of the embodiments disclosed herein, the cap secured by a frangible connection may be a securing component secured by a frangible connection. A cap is an element that may be resecured after breaking the frangible connection, e.g., by securing an engagement feature of the cap, e.g., an interior surface thereof, to an engagement feature of a lid or other component, e.g., an exterior surface of a lid, to which the cap had been frangibly secured. Exemplary engagement features include, e.g., threads, projections and notches, or combination thereof. A securing component is an element that, in some embodiments, may be resecured as for a cap, and, in other embodiments, is not configured to be resecured to the element to which the securing component had been frangibly secured.

### BRIEF DESCRIPTION OF THE DRAWINGS

FIG. 1 illustrates an embodiment of an extraction container of the invention;

FIG. 2 illustrates an exploded view of the extraction container of FIG. 1 with a foil seal covering a top opening of a tubular body of the extraction container having been removed from the top opening and a filter having been removed from within the tubular body;

FIG. 3 illustrates an assembly process for manufacturing the extraction container of FIG. 1;

FIG. 4 illustrates a process for using the extraction container of FIG. 1;

FIG. 5 illustrates another embodiment of an extraction container and a method of pooling samples of the invention; and

FIG. 6 illustrates a method for pooling samples of the invention.

### DETAILED DESCRIPTION

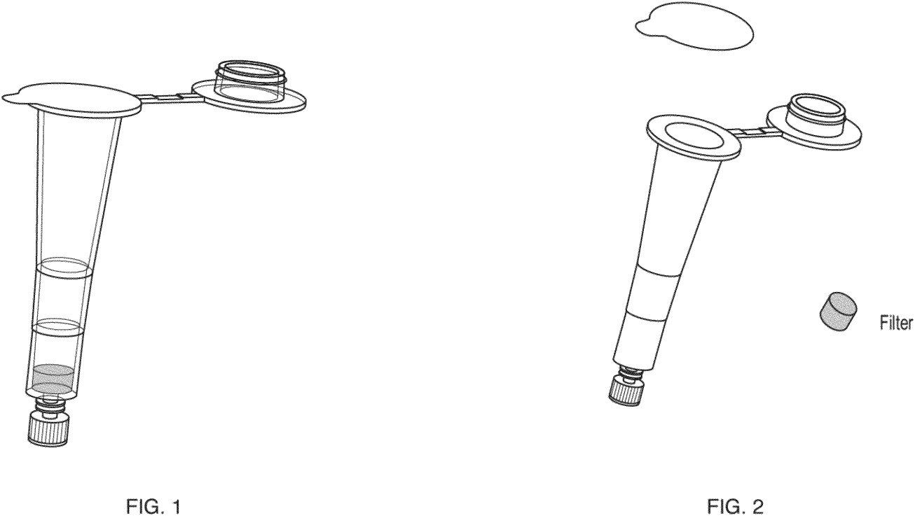

With reference to FIGS. 1 and 2, an extraction container includes a generally tubular body, a base cap disposed at a base of the tubular body, a seal obstructing a top opening at the top of the tubular body, a top cap connected to the tubular body via a hinge, and a filter disposed within the tubular body adjacent the base thereof. A liquid, such as an extraction buffer, is disposed within the tubular body. The generally tubular body, base cap, top cap and hinge are integrally formed, e.g., molded, and composed of a polymer material such as polypropylene, polyethylene, or cycloolefin.

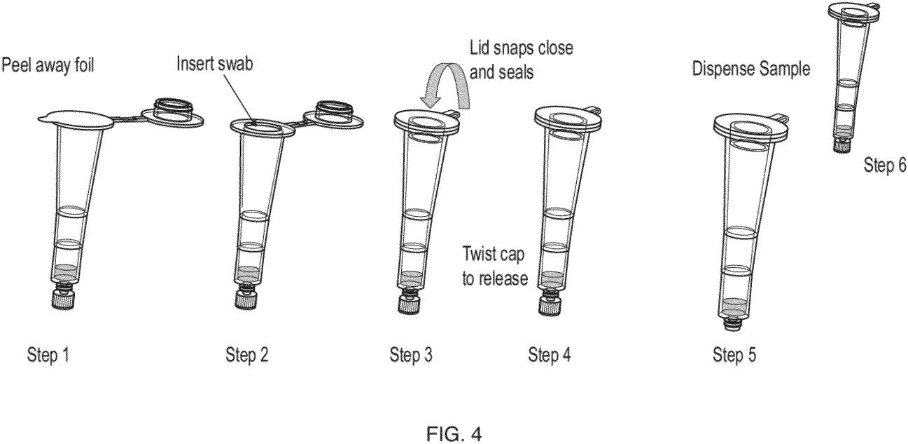

A proximal portion of the base cap is connected to the tubular body via a frangible connection. When connected to the tubular body via the frangible connection, the base cap obstructs a base opening disposed in the base of the tubular body and prevents the liquid from exiting the tubular body via the base opening. When the base cap is removed from the tubular body by breaking the frangible connection (see, e.g., the discussion below with respect to FIG. 4), liquid within the tubular body may be dispensed therefrom through the base opening. The exterior of the base of the tubular body and an interior of a distal portion of the base cap include corresponding engagement features, e.g., threads, projections and notches, or combination thereof, so that the base cap can be reattached to the tubular body by engaging the engagement features to again obstruct the base opening and prevent further exit of liquid from the tubular body (see, e.g., the discussion below with respect to FIG. 4).

The filter is typically composed of a polymeric material and configured to permit the passage of liquid and a target to be determined in a diagnostic determination but to obstruct the passage of concomitant materials that might degrade the performance, accuracy, and/or precision of the diagnostic determination. For example, the filter may be configured to obstruct the passage of fibers or other parts of a swab that may inadvertently be released into the extraction liquid within the tubular body. The filter is disposed within the tubular body, e.g., adjacent the base thereof, so that liquid exiting the tubular container through the base opening must first pass through the filter. The filter is disposed within the tubular body without requiring additional components, e.g., by press-fitting the filter in place.

The seal is typically composed of a metallic foil, a polymer layer, e.g., a polymer film, or combination thereof and is releasably secured to the top of the tubular body to prevent liquid from exiting the tubular body through the top opening. The seal prevents the passage of the liquid through the top opening as well as preventing the evaporation of the liquid therein. The seal may be heat staked to the tubular body. An exemplary seal is a multilayer seal having a thickness of about 0.025 mm and formed of polymer-metal-polymer layers, e.g., polyethylene-aluminum-polyethylene.

The top cap is secured to the tubular body via a hinge and may be used to re-obstruct the top opening after the seal has been removed to prevent liquid from exiting the tubular body through the top opening.

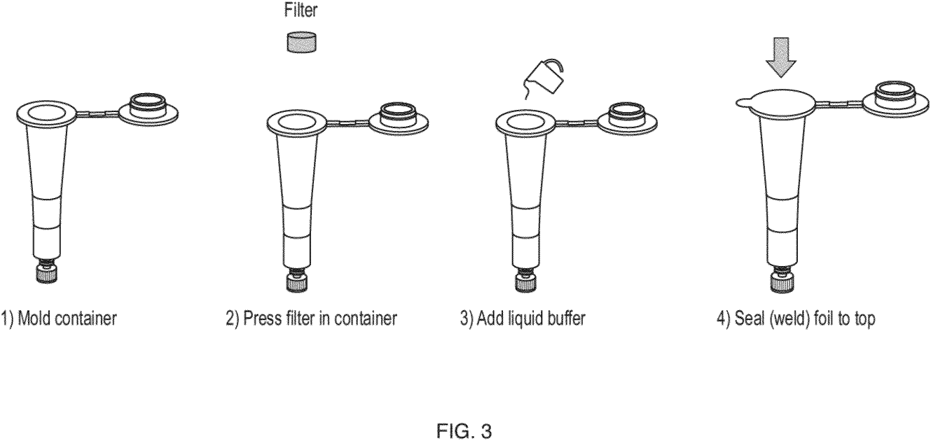

With reference to FIG. 3, a method for assembling the extraction container includes (1) integrally forming, e.g., by molding, the tubular body, base cap, top cap, and hinge, (1) pressing the filter into the tubular body, (3) adding an extraction liquid to the tubular body, and (4) sealing the top opening with the seal. In embodiments, the method for assembling the functional components of the extraction container consists essentially of the aforementioned steps. By functional components, it is meant the components of the extraction container in the form in which the extraction container is provided to an end user and which materially contribute to the functioning of the container in use. In a kit including such an extraction container, other components such as a swab, diagnostic cartridge, labels, or instructions would not be considered functional components of the extraction container itself.

With reference to FIG. 4, a method for using the extraction container includes (1) removing the seal from the top opening thereof, (2) inserting a sample collection device, e.g., a swab, carrying a biological sample into the liquid within the tubular body and manipulating the tubular body as by squeezing the exterior surface between the user’s fingers to extract some of the sample into the liquid to form a mixture, (3) removing the sample collection device and sealing the top opening with the top cap, (4) separating the base cap from the tubular body by breaking the frangible connection as by twisting the base cap, (5) dispensing some of the mixture through the filter and base opening, e.g., onto a sample application port of a diagnostic device, and (6) closing the base cap by engaging the engagement features of the base cap and tubular body. The mixture within the tubular body is then contained to prevent contamination of the user and their surroundings and also permits the mixture to be preserved for later analysis. The method of using the extraction container may further include subjecting the mixture to a heating step prior to the step of breaking the frangible connection of the cap. The heating may be performed using any of the techniques disclosed herein, e.g., by subjecting the container to microwave radiation. The heating may be performed to a temperature and for a duration sufficient to lyse cells of the biological sample and release contents therefrom. For example, the heating temperature and duration may be as disclosed herein. The heating step may be performed without removing the entirety of the sample collection device from the collection device. For example, the collection device may be a swab and the user may break a shaft of the swab thereby leaving a portion within the extraction container prior to sealing the top opening.

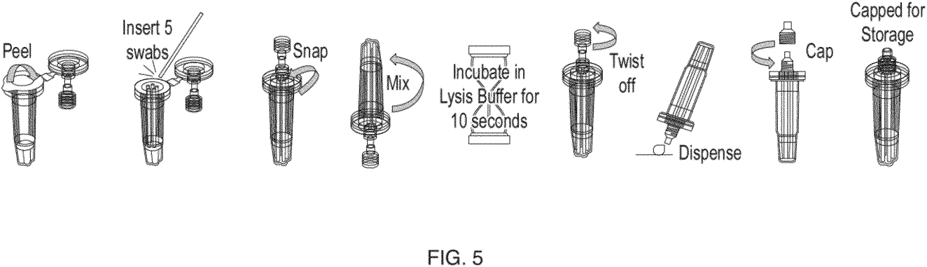

With reference to FIG. 5, an extraction container has a generally tubular body defining a longitudinal axis and including (i) a closed end and an open end spaced apart from one another along the longitudinal axis and (ii) an interior. A buffer liquid, e.g., VTM and/or a lysing medium, occupies a lower portion of the interior of the tubular body. The lower portion of the interior has an inner surface that is spaced apart radially from the longitudinal axis and defines a cinquefoil having five (5) lobes. A seal, e.g., as described for the embodiment of FIGS. 1 and 2, is releasably secured to the top of the open end of the tubular body to prevent liquid from exiting the tubular body through the opening during shipping and storage. The extraction container also includes a lid configured to close the open end. The lid has a passage configured, when in an open state, to permit liquid within the tubular body to pass through the passage and exit the tubular body. A cap is secured to the lid via a frangible connection and disposed to obstruct the passage, e.g., as described for the embodiment of FIGS. 1 and 2. When the cap is removed by breaking the frangible connection, the passage is opened. The cap and lid each include at least one respective engagement feature that can be engaged such that the cap again obstructs the passage, e.g., as shown in FIG. 4.

Continuing with FIG. 5, a method of using the extraction container to prepare a pooled sample may proceed as follows. A user removes the seal, e.g., by peeling, to expose the interior of the tubular member and the liquid disposed therein. The user provides a first swab that has a swab shaft and a collection swab tip at a distal end of the shaft. A portion of the swab shaft proximal to the collection swab tip is frangible to permit the shaft to be broken, separating the distal portion of the swab shaft from the proximal portion of the swab shaft. The collection swab tip will have been used to obtain a sample from a first subject, e.g., a first human being. The sample may be, e.g., a nasal or a nasopharyngeal sample. The first swab is inserted into the tubular member until at least a portion, substantially all, or essentially all of the collection swab tip is immersed in the liquid. The user then breaks the swab shaft of the first swab at the frangible connection so that the distal portion of the swab shaft including the collection swab tip is retained within the tubular member. A proximal portion of the swab shaft retained within the tubular member may include a portion of the frangible connection at which the shaft was broken.

The user then provides a second swab, the second swab having a collection swab tip that has been used to obtain a sample from a second, different subject, e.g., a second human being. Using the second swab, the user repeats the steps of inserting the swab into the liquid and breaking the frangible connection to leave the distal portion of the second swab in the liquid along with the distal portion of the first swab. The forgoing steps may repeated, e.g., an additional three times, each using a swab that has been used to obtain a sample from a respective different subject, e.g., a respective different human being. Typically, the samples from the different subjects are of the same type.

The cinquefoil interior of the extraction container includes five lobes, each of which at least partially accommodates a collection swab tip when inserted therein. Because of the lobed shape, the volume of liquid required to immerse the collection swab tips is less than would be required if the interior were generally circular. Accordingly, the liquid resulting from the pooling of the multiple samples will have a higher concentration of any targets. In addition, the lobes guide the user when inserting successive swabs and provide a visual indicator of when five swabs have been inserted.

The position of the frangible portion of the swab shafts is chosen so that lid can be closed even with the distal portions of the swabs retained within the tubular body. The user closes the lid and then mixes the liquid, which forms a mixture including five pooled samples. After mixing and optional incubation, the user breaks the cap from the lid, dispenses an amount of sample for analysis, and then uses the cap to once again seal the pooled sample liquid within the extraction container. The method may further include subjecting the mixture with pooled samples to a heating step prior to the step of breaking the frangible connection of the cap. The heating may be performed using any of the techniques disclosed herein, e.g., by subjecting the container to microwave radiation. The heating may be performed to a temperature and for a duration sufficient to lyse cells of the biological sample and release contents therefrom. For example, the heating temperature and duration may be as disclosed herein.

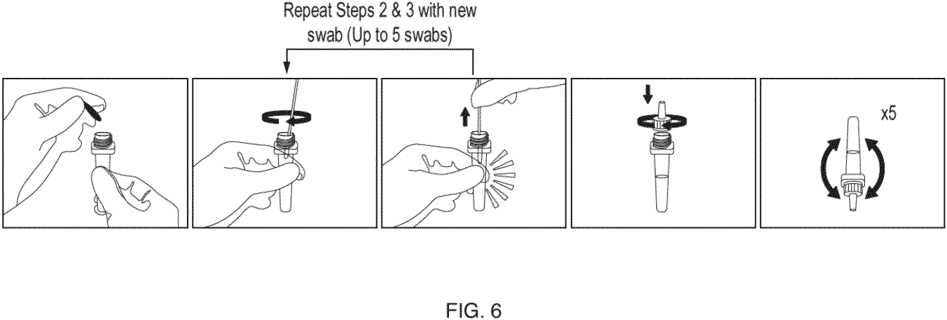

The cinquefoil is a multifoil that has a number N = five lobes. As an alternative, the extraction container may have an interior with a multifoil that is, e.g., a trefoil (3 lobes), quatrefoil (4 lobes), sexfoil (6 lobes), septfoil (7 lobes), or octofoil (8 lobes). The interior surface of each lobe may be, for example, arcuate (e.g., circular or elliptical) or linear (e.g., wedge”<” shaped). Turning to FIG. 6, a method of using an extraction container to prepare a pooled sample may proceed as follows. The extraction container includes an extraction buffer disposed therein and a seal, as disclosed herein. A user removes the seal, e.g., by peeling, to expose the interior of the tubular member of the extraction container and the liquid disposed therein. The user provides a first swab that has a swab shaft and a collection swab tip at a distal end of the shaft. The collection swab tip will have been used to obtain a sample from a first subject, e.g., a first human being. The sample may be, e.g., a nasal or a nasopharyngeal sample. The first swab is inserted into the tubular member until at least a portion, substantially all, or essentially all of the collection swab tip is immersed in the liquid. The user then agitates or stirs the swab within the liquid for a period of time, e.g., 10 seconds, to combine the sample with the liquid. The user then removes the first swab from the extraction container, squeezing the exterior of the extraction vial, e.g., between the user’s fingers, to compress the collection swab tip during removal to extract liquid absorbed thereon.

Once the first swab has been removed from the extraction container, the user provides a second swab, the second swab having a collection swab tip that has been used to obtain a sample from a second different subject, e.g., a second human being. Using the second swab, the user repeats the steps of inserting the swab into the liquid and agitating or stirring the swab to combine the sample of the second swab with the liquid and first sample within the extraction container. The forgoing steps may repeated, e.g., an additional three times, each using a swab that has been used to obtain a sample from a respective different subject, e.g., a respective different human being. Typically, the samples from the different subjects are of the same type. The resulting liquid in the extraction container is a mixture including at least some of the sample from each of the swabs. A portion of the mixture may be removed from the extraction container as disclosed herein, e.g., by breaking the frangible connection of the cap to expose a dispensing opening, and subjected to an assay for the determination of one or more targets therein. The method may further include subjecting the mixture to a heating step prior to the step of breaking the frangible connection of the cap. The heating may be performed using any of the techniques disclosed herein, e.g., by subjecting the container to microwave radiation. The heating may be performed to a temperature and for a duration sufficient to lyse cells of the biological sample and release contents therefrom. For example, the heating temperature and duration may be as disclosed herein.

In embodiments, the functional components of an extraction container as disclosed herein consist essentially of the integrally formed, e.g., molded, tubular body and base cap. In some embodiments, the functional components of the extraction container further consist essentially of a filter disposed within such tubular body with an integrally formed first cap, which may be at the base or top of the extraction container. In still further embodiments, the functional components of the extraction container further consist of the liquid disposed within such tubular body, a seal covering a top opening of the tubular body, and, optionally, a second cap and hinge integrally formed with the tubular body and first cap, where the second cap is configured to reseal the top opening of the tubular body after removing the seal.

In embodiments, an extraction container consists of the following functional components: an integrally formed, e.g., molded, tubular body with base cap connected to the tubular body via a frangible connection, a filter disposed within the tubular body and configured such that liquid exiting a base opening of the tubular body must pass through the filter, an extraction liquid disposed within the tubular body, and a seal covering a top opening of the tubular body. In embodiments the integrally formed tubular body and base cap further include a top cap connected to the tubular body via a hinge with the top cap and hinge being integrally formed with the tubular body and base cap.

A tubular or generally tubular body may define a cross section about a longitudinal axis. The cross section may be, e.g., arcuate such as circular or elliptical, and/or include linear features such as a polygonal cross section. A multifoil may include arcuate and/or linear features.

In embodiments, a kit includes any of the extraction containers as disclosed herein, a sample collection device such as a swab, and a diagnostic cartridge such as a microfluidic device, and methods for using same, as described in U.S. Provisional Application No. 62/991,446 filed Mar. 18, 2020 or U.S. Provisional Application No. 63/067,782 filed Aug. 19, 2020 (the ‘782 application) and International Patent Application No. PCT/US2021/013325 (“the ‘325 Application”) titled “Fluid Control in Microfluidic Devices” and filed Jan. 13, 2021, and/or diagnostic reagents and methods for determination of a pathogen as described in U.S. Provisional Application No. 62/992,681 filed Mar. 20, 2020, International Patent Application No. PCT/US2021/023532, filed Mar. 22, 2021, and/or the ‘325 application. Each of these applications is incorporated herein by reference in its entirety. The extraction container may be used to apply a mixture of extraction buffer and sample to the diagnostic cartridge to permit the determination of a target, e.g., a pathogen or target indicative of the presence of a pathogen, in the sample. In some embodiments, the kit includes an extraction container and a number N collection swabs, where N is at least the number of samples to be pooled using the extraction container. In embodiments in which the extraction container includes a multifoil, the number N of swabs may be the same as the number of the multifoil.

An immunological assay may be performed using a sample that is a mixture including at least a portion of a sample obtained from each of a plurality of subjects, e.g., mammals. Each of the subjects or mammals may be a human being. A determination of the presence of a pathogen in the mixture is indicative that the pathogen was present in the sample obtained from at least one of the plurality of subjects. A determination of the absence of a pathogen in the mixture is indicative that the pathogen was absent from each of the samples obtained from each of the plurality of subjects. Such a multi-subject assay can be used, for example, to reduce the total number of assays required to surveil the presence of an infectious disease in a population of subjects.

The immunological assay may be an assay for the determination of a target, e.g., a biomolecule such as an antigen, antibody, or protein, indicative of the presence of an infectious disease. For example, the assay may be any of the assays disclosed or claimed in the ‘325 application, e.g., as in any of claims 124-139 or 176-214 thereof, in which the “sample” is a mixture comprising samples from each of the plurality of subjects. The assay may be performed using any of the microfluidic devices and systems disclosed or claimed in the ‘325 application, e.g., in any of claims 140-156.

The number of subjects “N” may be, for example, at least about 2, at least about 3, at least about 4, at least about 5, at least about 6, at least about 7, at least about 8, at least about 9, or at least about 10. The number “N” may be, for example, about 25 or less, about 20 or less, about 15 or less, about 10 or less, about 9, or less, about 8, or less, about 7 or less, about 6 or less, or about 5 or less.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method comprising:
removing a removable seal sealing an access opening of a generally tubular body thereby exposing a liquid disposed within the generally tubular container;
combining a sample comprising a target with the exposed liquid thereby forming a liquid mixture of the sample and the liquid within the generally tubular body;
resealing the access opening with a lid comprising (i) a dispensing opening and (ii) a securing component that obstructs the dispensing opening and is secured to the lid via a frangible connection;
with the access opening resealed with the lid, heating the liquid mixture within the generally tubular container to a temperature of at least about 100° C., wherein the lid and the securing component essentially prevent liquid of the liquid mixture or gas within the generally tubular body from leaking or escaping therefrom during the heating; and
after heating the liquid mixture, opening the dispensing opening by breaking the frangible connection between the securing component and the lid and then dispensing at least some of the liquid mixture from the generally tubular body through the dispensing opening.

2. The method of claim 1, wherein the generally tubular container and the lid including the securing component are made of a polymer.

3. The method of claim 1, wherein the securing component is a cap and the method further includes resecuring the cap to the lid to reseal the dispensing opening after dispensing the at least some liquid mixture.

4. The method of claim 1, wherein the method further includes subjecting the at least some dispensed liquid mixture to an assay to determine the presence and/or amount of the target.

5. The method of claim 4, wherein the target is indicative of a pathogen.

6. The method of claim 1, wherein the step of heating comprises subjecting the liquid mixture within the generally tubular body to microwave radiation.

7. The method of claim 1, wherein the step of heating comprises heating the liquid mixture to a temperature and for a duration sufficient to lyse bacterial cells within the liquid mixture.

8. The method of claim 7, wherein the step of heating comprises heating the liquid mixture to a temperature and for a duration sufficient to lyse bacterial cells of Mycobacterium tuberculosis and/or a bacterium of the genus Streptococcus.

9. The method of claim 8, wherein the target is indicative of at least one of Mycobacterium tuberculosis and/or a bacterium of the genus Streptococcus and the method further includes subjecting the at least some dispensed liquid mixture to an assay to determine the presence and/or amount of the target.

10. The method of claim 1, wherein the heating step comprises heating the liquid mixture for a period of less than about 200 seconds and more than about 60 seconds.

11. The method of claim 1, wherein the heating step comprises heating the liquid mixture to a temperature that is at least about 115° C. and less than about 150° C.

12. The method of claim 1, wherein the total volume of the liquid mixture within the tubular body during the heating step is less than about 50 µL.

13. The method of claim 1, wherein, during the heating step, no more than about 0.5% of the total mass of the liquid mixture present at the start of the heating step escapes or leaks from the tubular body.

14. The method of claim 13, wherein, during the heating step, less than about 0.25% of the total mass of the liquid mixture present at the start of the heating step escapes or leaks from the tubular body.

15. The method of claim 1, wherein the step of dispensing comprises squeezing an exterior surface of the tubular body between fingers of a user to increase a gas pressure therein and dispense the at least some liquid mixture through the dispensing opening.

16. The method of claim 1, wherein the lid comprises a filter and the step of dispensing comprises passing the at least some liquid mixture through the filter.

17. The method of claim 1, wherein the liquid within the tubular body comprises at least one of buffer, universal transport media (UTM), Viral Transport Media (VTM) or lysis medium.

18. The method of claim 1, wherein the sample comprises at least one of saliva, mucous, sputum, or blood.

19. The method of claim 1, wherein the removable seal comprises one or more layers comprising a liquid-impermeable metal foil and/or a liquid-impermeable polymer film.

20. The method of claim 1, wherein an interior surface of the tubular body defines a multifoil.
